(19) United States
(12) Patent Application Publication
(10) Pub. No.: US 2024/0024131 A1
(43) Pub. Date: Jan. 25, 2024

(54) SURGICAL INSTRUMENT, FEMORAL TRIAL IMPLANT AND SURGICAL INSTRUMENT SYSTEM

(71) Applicant: Aesculap AG, Tuttlingen (DE)

(72) Inventors: Franz-Peter Firmbach, Emmingen-Liptingen (DE); Lilli Stoffels, Calw (DE)

(21) Appl. No.: 18/347,900

(22) Filed: Jul. 6, 2023

(30) Foreign Application Priority Data
Jul. 25, 2022 (DE) 10 2022 207 577.3

(51) Int. Cl. A61F 2/46; A61F 2/38; A61F 2/30

(52) U.S. Cl. CPC A61F 2/4684; A61F 2/3859; A61F 2/30749

(57) ABSTRACT

A surgical instrument for use in a knee joint replacement operation includes a fastening device configured for releasably fastening to a femoral trial implant mounted on a distal femur. The fastening device has two positioning pins arranged spaced apart mediolaterally from each other and which, for the purpose of positioning the fastening device, are designed to be able to be plugged in a posterior direction into two receiving bores, complementary to the positioning pins, of the trial implant. The fastening device has at least one fixing element for fixing the fastening device in a form-fitting manner to the trial implant in an anterior direction.

### CROSS-REFERENCE TO RELATED APPLICATION(S)

This application claims priority under 35 U.S.C. § 119 to German Application No. 10 2022 207 577.3, filed on Jul. 25, 2022, the content of which is incorporated by reference herein in its entirety.

### FIELD

The present disclosure relates to a surgical instrument for use in a knee joint replacement operation. The present disclosure also relates to a femoral trial implant for use in a knee joint replacement operation. The present disclosure further relates to a surgical instrument system for use in a knee joint replacement operation.

### BACKGROUND

In a knee joint replacement operation, or total knee arthroplasty (TKA), joint surfaces of the femur and/or tibia that have become worn or have been otherwise affected by disease or by injury are replaced by artificial joint surfaces of a knee joint prothesis. Such knee joint protheses usually comprise a femoral component and a tibial component. The femoral component is implanted at the distal end of the femur. The tibial component is implanted at the proximal end of the tibia.

Before the implantation of the prosthetic components, the distal femur and the proximal tibia are resected. For this purpose, the surgeon makes various resection cuts and detaches bone and/or cartilage material from the respective bone. The resection enables the respective bone to be adapted in its shape to the prosthetic component that is to be received.

The resection can be carried out on the basis of different concepts. One concept aims to keep the tension of the ligaments of the knee balanced during the movement of the joint. This is intended to ensure better functioning of the knee joint prosthesis. This concept is generally referred to as “gap balancing”. In other concepts, the surgeon uses the resection to remove a certain amount of bone and/or cartilage material. Such concepts are generally referred to as “measured resection”. The alignment of the resection cuts with respect to the patient's anatomy determines the subsequent alignment of the implanted components and consequently also the orientation of the prosthetic joint axes. The alignment of the resection cuts is therefore of particular importance.

In the alignment of the resection cuts, there are mainly three approaches: mechanical, anatomical and kinematic. Mechanical alignment involves resection of the proximal tibia perpendicularly to the longitudinal axis of the tibial shaft. The resection of the distal femur takes place in a manner correspondingly adapted thereto. If so required, ligament releases are performed. The anatomical alignment attempts to resect the tibia at a varus angle of 3°. Femoral resection and ligament releases are performed with the aim of obtaining a straight hip-knee-ankle axis of the leg. The goal of kinematic alignment is to implant the artificial joint surfaces of the prosthetic components at the level of the natural, defect-free joint surface present prior to the development of arthritis.

In kinematic alignment, the alignment of the resection cuts often starts on the distal femur. The resection of the proximal tibia is adapted thereto. For the purpose of resecting femur and tibia one after the other in time, special surgical instruments are known which are also designated as tibial cut alignment guides. Such instruments allow the alignment of the femoral resection cuts to be transferred to the tibia. The transfer usually takes place after an at least distal resection of the femur, in which the distal condyles are detached. The transfer can take place in extension or flexion. In a variant of kinematic alignment, the distal femur is first of all completely prepared (all femur first). The resection cuts to be made on the distal femur are aligned with respect to the patient's anatomy and applied to the distal femur. A femoral trial implant is then applied to the distal femur. The femoral trial implant already aligned according to the patient's anatomy is then used as a reference component, the alignment of which is transferred by means of the transfer tool to the proximal tibia, in order to align and apply the resection cuts that are to be made on the latter. It will be appreciated that, as an alternative, a procedure in the reverse order is conceivable, i.e. starting from the proximal tibia.

Said transfer tools usually have a fastening device which is configured for releasable fastening to a femoral trial implant. Typically, the fastening device can be releasably fastened to the trial implant in a form-fitting manner. Such trial implants are often designed with thin walls. Only a relatively small material thickness is therefore available for producing the form fit. Consequently, producing and reliably maintaining the form fit proves to be particularly challenging. The often small material thickness of the trial implant can result in elastic and/or plastic deformations. These can cause loosening and/or loss of the form fit. Moreover, tilting movements of the fastening device relative to the trial implant can occur. These can distort the alignment of the transfer tool.

### SUMMARY

The object of the present disclosure is to make available a surgical instrument, a femoral trial implant and a surgical instrument system which afford advantages over conventional surgical instruments, femoral trial implants and surgical instrument systems. A particular object is to allow the surgical instrument to be fastened to the trial implant in a manner particularly secure against tilting.

The surgical instrument according to the present disclosure serves for use in a knee joint replacement operation and has: a fastening device which is configured for releasably fastening to a femoral trial implant mounted on a distal femur, wherein the fastening device has two positioning pins which are arranged spaced apart mediolaterally from each other and which, for the purpose of positioning the fastening device, are designed to be able to be plugged in a posterior direction into two receiving bores, complementary to the positioning pins, of the trial implant, wherein the fastening device has at least one fixing element for fixing the fastening device in a form-fitting manner to the trial implant in an anterior direction. The trial implant is not a constituent part of the surgical instrument. By interaction of the positioning pins with the receiving bores, the surgical instrument can be positioned mediolaterally, anteroposteriorly and proximodistally in a correct alignment relative to the trial implant. By means of the fixing element, the positioning of the surgical instrument can be fixed in a form-fitting manner. The fixing element counteracts undesired release of the plug connection. It specifically counteracts an anterior sliding movement of the positioning pins out of the receiving bores. The fixing element advantageously ensures that the positioning pins remain at a sufficient depth in the receiving bores. A surface pressure arising between the positioning pins and the receiving bores can therefore be minimized. A deformation of the positioning pins and/or of the receiving bores can also be minimized accordingly when a tilting torque acts between the fastening device and the trial implant. As a result, an undesired tilting of the surgical instrument relative to the trial implant is therefore counteracted particularly effectively. Accordingly, the surgical instrument proves particularly reliable and robust in its use. Moreover, it permits particularly precise transfer of the alignment of the trial implant to a proximal tibia.

The position and direction designations used in this description relate to the body of a patient, in particular the femur of the patient, and are to be understood in accordance with their usual anatomical meaning. Consequently, “anterior” denotes front or lying to the front, “posterior” denotes rear or lying to the rear, “medial” denotes inner or lying to the inside, “lateral” denotes outer or lying to the outside, “proximal” denotes towards the centre of the body, and “distal” denotes away from the centre of the body. Furthermore, “proximodistal” denotes along, preferably parallel to, a proximal-distal axis, “anteroposterior” denotes along, preferably parallel to, an anterior-posterior axis, and “mediolateral” denotes along, preferably parallel to, a medial-lateral axis. The aforementioned axes are orthogonal to one another and can of course be understood in relation to X, Y and Z axes not associated with the anatomy of the patient. For example, the proximal-distal axis can be designated alternatively as the X axis. The medial-lateral axis can be designated as the Y axis. The anterior-posterior axis can be designated as the Z axis. For the sake of better illustration and simplicity of the designations, the aforementioned anatomical position and direction designations are primarily used in the following.

In one embodiment of the present disclosure, the fixing element has at least one form-fitting portion for producing a form fit between the fastening device and the trial implant. The form-fitting portion can comprise a lug, a hook, a pin, a screw or the like. This permits particularly reliable fixing of the surgical instrument to the femoral trial implant.

In a further embodiment of the present disclosure, the fixing element comprises at least one snap-in hook. Such a snap-in hook can automatically latch onto the trial implant when the positioning pins are inserted into the complementary receiving bores. It is thus advantageously possible to dispense with a separate locking measure for producing the form fit when mounting the surgical instrument on the femoral trial implant. Accordingly, the surgical instrument proves particularly easy to handle. It also helps to keep short the duration of an operation.

In a further embodiment of the present disclosure, the fixing element is adjustably movable relative to the positioning pins between a fixing position and a release position. In the fixing position, the form fit can be produced. In the release position, the form fit can be cancelled. Preferably, the fixing element can be moved manually between its release position and its fixing position. Thus, the form fit can if appropriate be cancelled by moving the fixing element to its release position in order to separate the surgical instrument from the trial implant. Preferably, the form fit is cancelled purely manually and/or without use of a separate tool.

In a further embodiment of the present disclosure, the fastening device comprises at least one spring element, in particular a torsion or beam spring element, for generating a spring force which biases the fixing element in the direction of its fixing position. A movement of the fixing element to its release position can thus take place counter to and by overcoming the spring force. Inadvertent release of the form fit can thus be particularly effectively avoided. Moreover, the fixing element can be automatically moved to its fixing position by the spring force and held there. The form fit can thus be produced automatically when the positioning pins are inserted into the receiving bores. Separate actuation of the fixing element by the operator is in this case rendered obsolete.

In a further embodiment of the present disclosure, the positioning pins are each elongate in an anteroposterior direction between a first and a second end, wherein a first end portion present at the respective first end is receivable in the posterior direction in an associated one of the receiving bores of the trial implant so as to form a plug connection. Positioning pins of this kind can be produced particularly cost-effectively, in particular with sufficient precision.

In a further embodiment of the present disclosure, the form-fit portion creates an undercut with respect to the anterior direction. The undercut prevents accidental separation of the fastening device from the trial implant in the anterior direction. This permits particularly reliable fixing of the fastening device.

In a further embodiment of the present disclosure, the first ends are exposed posteriorly and the undercut is exposed anteriorly, such that the trial implant can be positioned, in particular clamped, anteroposteriorly between the first ends on the one hand and the undercut on the other hand. In other words, the first ends and the undercut are oriented counter to one another in the anteroposterior direction. Thus, the position of the fastening device can advantageously be fixed both anteriorly and posteriorly.

The present disclosure further relates to a femoral trial implant for use in a knee joint replacement operation. It has: a bone contact surface which is configured for contacting a distal femur, two receiving bores which are each configured to receive a positioning pin of a surgical instrument, in particular of one according to the present disclosure and according to the above description, and a fixing portion which is configured for form-fit connection to a fixing element of the surgical instrument. The abovementioned advantages of the surgical instrument according to the present disclosure also apply mutatis mutandis to the femoral trial implant according to the present disclosure.

In one embodiment of the present disclosure, the bone contact surface is arranged on a proximal rear face of a main body of the trial implant and lies opposite a distal front face of the main body, wherein the main body has two condyle portions which are spaced mediolaterally apart from each other and which each extend in the anteroposterior direction and transition at their anterior ends into a connection portion of the main body, wherein the fixing portion is arranged at a posterior end of the connection portion, mediolaterally between the two condyle portions. This results in a central arrangement of the fixing portion. The fixing portion can be arranged in a region in which the intercondylar notch was present before the resection of the femur. The fixing portion arranged in this way allows a holding force to be introduced centrally from the fixing element into the trial implant. This ensures a particularly stable releasable connection of fastening device and trial implant in a manner secure against tilting.

In a further embodiment of the present disclosure, the fixing portion is arranged mediolaterally between the two receiving bores and, alternatively or additionally, at a proximodistal distance from the two receiving bores. The mediolateral arrangement ensures particularly uniform introduction of fastening forces into the trial implant via the form-fitting element and the positioning pins. The proximodistal distance between the fixing element and the receiving bores can advantageously function as a lever arm for supporting torques between fastening device and trial implant.

In a further embodiment of the present disclosure, at least one of the receiving bores, preferably each of them, is designed as a blind hole. A bottom of the blind-hole-shaped receiving bores advantageously forms an in particular posterior stop for the positioning pins. Accordingly, a posterior position of the surgical instrument relative to the trial implant can be fixed in a particularly simple way and permanently.

The present disclosure further relates to a surgical instrument system for use in a knee joint replacement operation, having: a surgical instrument according to the present disclosure and in accordance with the above description, a femoral trial implant according to the present disclosure and in accordance with the above description, wherein the surgical instrument is fastened releasably to the trial implant, wherein the positioning pins and the receiving bores are designed complementing each other, and the positioning pins are at least partially plugged into the receiving bore along the posterior direction, wherein the fixing element and the fixing portion are tailored to each other to produce a form fit, and the surgical instrument is fixed on the trial implant by means of the form fit, in particular in the anterior direction. The abovementioned advantages of the surgical instrument according to the present disclosure and of the femoral trial implant according to the present disclosure also apply mutatis mutandis to the surgical instrument system according to the present disclosure.

In a further embodiment of the present disclosure, the surgical instrument system comprises a plurality of femoral trial implants of different sizes, wherein, in all of the femoral trial implants, both of the receiving bores are arranged, particularly with respect to their central longitudinal axes, at an identical proximodistal distance from a proximally outermost point of the respective femoral trial implant, wherein the receiving bores of each of the femoral trial implants are arranged at an identical mediolateral distance from each other, in particular with respect to the central longitudinal axes. This expressly does not mean that the proximodistal distance and the mediolateral distance have to be identical in extent in one and the same trial implant. Instead, in the plurality of trial implants of different sizes, both the proximodistal distance and the mediolateral distance are the same regardless of said size. In other words, all of the trial implants of the surgical instrument system have a uniform proximal distance and a uniform mediolateral distance. Advantageously, one and the same surgical instrument can therefore be used with trial implants of different sizes. The sizes of the trial implants can be tailored to differently sized femoral bones. Accordingly, from the plurality of trial implants, it is possible to choose a trial implant of a certain size that best suits a given anatomy of the patient. Independently of the size of the trial implant, the alignment of the trial implant on the anatomy of the patient can be transferred to the tibia by means of the surgical instrument. In particular, a correct tibial cutting height is obtained independently of the chosen size of the trial implant. The proximally outermost point can also be referred to as the dwell point.

### BRIEF DESCRIPTION OF THE DRAWINGS

Further advantages and features of the present disclosure will become clear from the following description of preferred exemplary embodiments of the present disclosure which are shown in the drawings.

It will be appreciated that the features mentioned above and those still to be explained below can be used not only in the respectively indicated combination but also in other combinations or singly, without departing from the scope of the present disclosure.

FIG. 1 shows a schematic perspective view of an embodiment of a surgical instrument system according to the present disclosure with a surgical instrument, designed according to the present disclosure, in an intraoperative situation,

FIG. 2 shows a detail B of the surgical instrument system according to FIG. 1,

FIG. 3 shows the surgical instrument system according to FIGS. 1 and 2 in a schematic perspective view from another angle of observation,

FIG. 4 shows a schematic front view of the surgical instrument system according to FIGS. 1 to 3,

FIG. 5 shows a schematic sectional view along a section line D-D according to FIG. 4,

FIG. 6 shows a detail H of the view according to FIG. 5,

FIG. 7 shows a schematic sectional view along a section line G-G according to FIG. 4,

FIG. 8 shows a schematic perspective view of a further embodiment of a surgical instrument system according to the present disclosure with a further surgical instrument designed according to the present disclosure.

### DETAILED DESCRIPTION

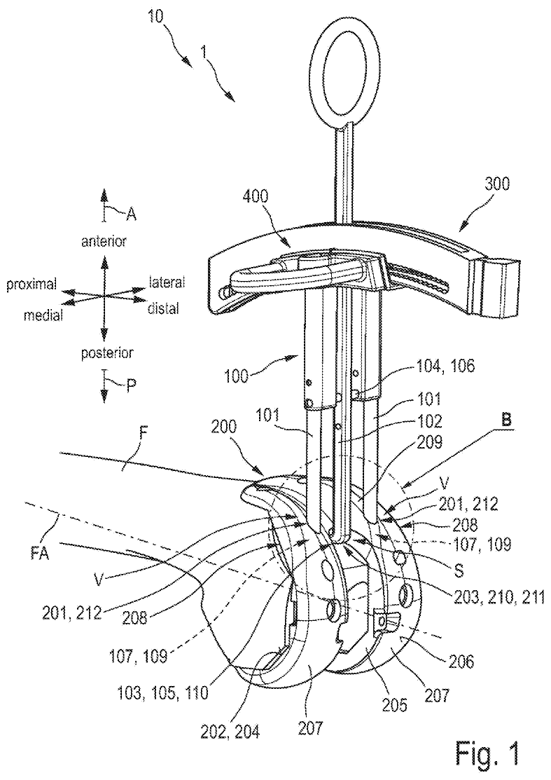

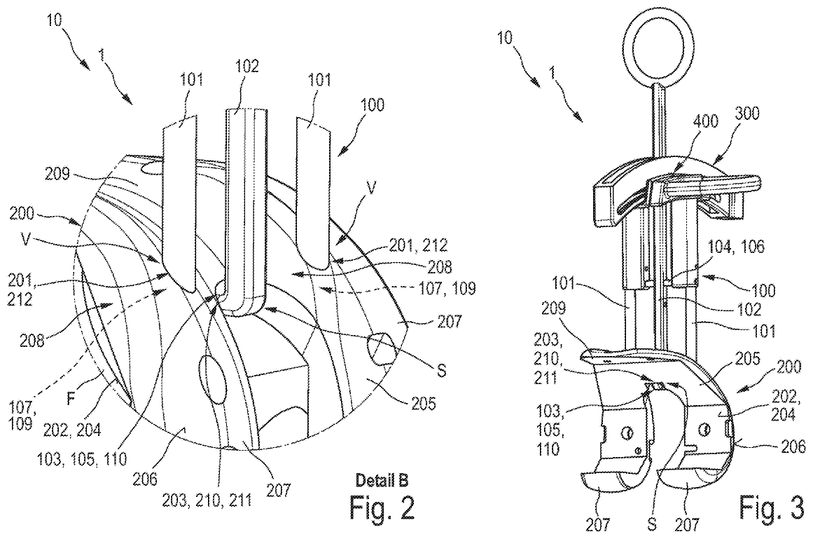

According to FIGS. 1 and 2, a surgical instrument 1 is provided for use in a knee joint replacement operation. The surgical instrument 1 can also be designated as a tibia transfer tool and serves for referenced positioning of a tibial cutting block on a proximal tibia. The tibial cutting block and tibia are not shown in the figures. A femoral trial implant 200 already positioned on a distal femur F serves as a reference for the position of the tibial cutting block. The femoral trial implant 200 and the tibial cutting block are not constituent parts of the surgical instrument 1. The trial implant 200 and the surgical instrument 1 are constituent parts of a surgical instrument system 10. The surgical instrument 1 and the surgical instrument system 10 are suitable in particular for knee joint replacement operations which follow the all femur first approach. In this approach, all of the required femoral resection cuts are made first. After resection of the distal femur F, the femoral trial implant 200 is fastened, and the latter can then function as femoral reference component for the surgical instrument 1. In FIGS. 1 and 2, the femoral trial implant 200 is fastened to the previously resected distal femur F. By means of the surgical instrument 1, an alignment of the trial implant 200 already made on the anatomy of the patient can be transferred to a proximal tibia, in order there to make at least one resection cut in the correct alignment.

The surgical instrument 1 has a fastening device 100. The fastening device 100 is configured for releasable fastening to the femoral trial implant 200. The fastening device 100 comprises two positioning pins 101. The two positioning pins 101 are arranged spaced apart at a mediolateral distance from each other. The positioning pins 101 are designed to be able to be plugged into two receiving bores 201 of the trial implant 200 for the purpose of positioning the fastening device 100. The receiving bores 201 are designed complementing the positioning pins 101. The positioning pins 101 are designed to be able to be plugged in a posterior direction P into the receiving bores 201. In other words, the positioning pins 101 can be inserted or pushed along the posterior direction P into the complementary receiving bores 201.

The fastening device 100 further comprises at least one fixing element 102. The fixing element 102 serves to hold and/or fix the fastening device 100 on the trial implant 200 in a form-fitting manner. The fixing element 102 is configured to form-fittingly hold and/or fix the fastening device 100 on the trial implant 200 in an anterior direction A. The fixing element 102 has at least one form-fitting portion 103. The form-fitting portion 103 serves to produce a form fit S between fastening device 100 and trial implant 200. The form-fitting portion 103 can be designed in the form of a lug, a hook, a pin, a screw or similar. In the present case, the fixing element 102 comprises a snap-in hook 105. The snap-in hook 105 automatically latches onto the trial implant 200 when the two positioning pins 101 are plugged into the receiving bores 201.

The fixing element 102 is adjustably movable, relative to the positioning pins 101, between a fixing position and a release position. The form fit S can be produced in the fixing position. The form fit can be cancelled or released in the release position. In the intraoperative situation shown in FIGS. 1 and 2, the fixing element 102 is located in its fixing position. Here, the surgical instrument 1 is fixed on the trial implant 200 by means of the fixing element 102. By movement of the fixing element 102 to its release position, the surgical instrument 1 can be separated in the anterior direction A from the trial implant 200.

The fastening device 100 moreover has at least one spring element 106. The spring element 106 in the present case is a torsion spring element 104. The spring element 106 serves to generate a spring force, which biases the fixing element 102 in the direction of its fixing position. The fixing element 102 is thus held in its fixing position by means of the spring force. A movement of the fixing element 102 to its release position is obtained by overcoming the spring force. On account of the spring force, the fixing element 102 can latch automatically in a form-fitting manner onto the trial implant 200 when the positioning pins 101 are plugged in. In other words, the spring force in the present case has the effect that the fixing element 102 latches automatically onto the trial implant 200 when the positioning pins 101 are plugged in.

The positioning pins 101 are each elongate in the anteroposterior direction between a first and a second end 107, 108. A first end portion 109 is present at the first end 107. The first end portion 109 is able to be received in the posterior direction P in an associated bore of the receiving bores 201 of the trial implant 200, so as to form a plug connection V. At least one of the positioning pins 101 and its associated receiving bore 201 can be tailored to each other in the manner of a narrow clearance fit or a transition fit. If one of the positioning pins 101 and its associated receiving bore 201 are tailored to each other with a precise fit, the respective other positioning pin 101 and its associated receiving bore 201 can have mediolateral play. For this purpose, the receiving bore 201 concerned can be designed as an oblong hole in order to generate the mediolateral play. In the present case, the positioning pins 101 have a circular cross section at least in the region of their first end portion 109. It will be appreciated that, as an alternative, other cross-sectional shapes are also conceivable, for example in the form of an oval or of a rectangle or of another polygon.

In the present case, the form-fitting portion 103 creates an undercut 110 with respect to the anterior direction A. The first ends 107 are exposed posteriorly. The undercut 110 is exposed anteriorly. The first ends 107 and the undercut 110 are oriented counter to each other in the anteroposterior direction. As a result, the trial implant 200 is able to be positioned anteroposteriorly between the first ends 107 on the one hand and the undercut 110 on the other hand. The undercut 110 can form an abutment for the positioning pins 101.

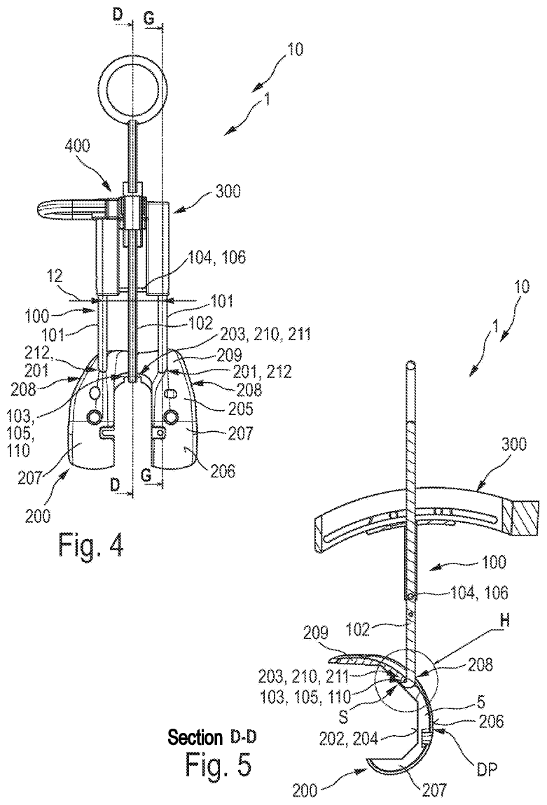

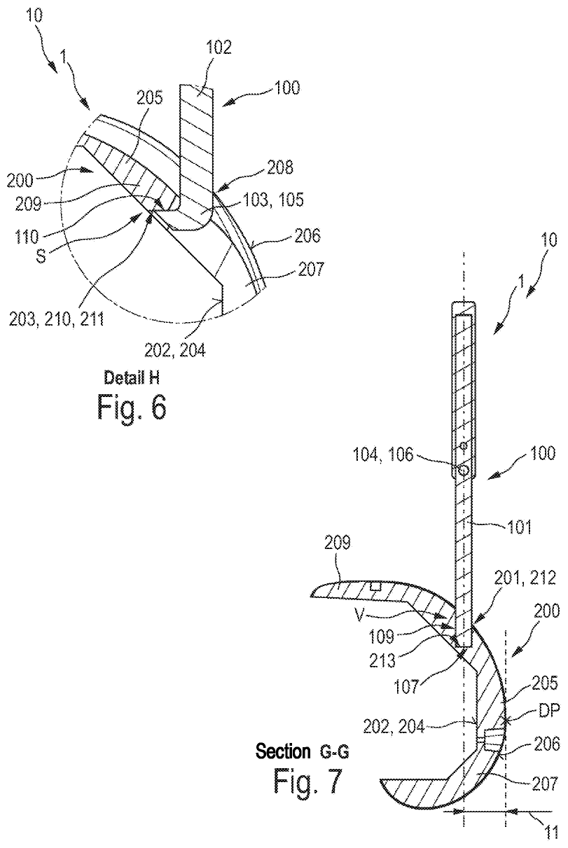

FIGS. 1 to 8 show an embodiment of a femoral trial implant 200 according to the present disclosure. The femoral trial implant 200 is provided for use in a knee joint replacement operation. It has a bone contact surface 202. The bone contact surface 202 is configured for contacting the distal femur F. The femoral trial implant 200 moreover comprises the two receiving bores 201. These are each configured to receive one of the positioning pins 101 of the surgical instrument 1. The femoral trial implant 200 moreover comprises a fixing portion 203. The fixing portion 203 is configured for form-fit connection to the fixing element 102 of the surgical instrument 1. In the present case, the femoral trial implant 200 has an in particular claw-shaped main body 205. The bone contact surface 202 is arranged on a proximal rear face 204 of the main body 205. The proximal rear face 204 lies opposite a distal front face 206 of the main body 205. The main body 205 comprises two condyle portions 207 spaced mediolaterally apart from each other. The two condyle portions 207 each extend in the anteroposterior direction. At their anterior ends 202, the condyle portions 207 transition into a connection portion 209 of the main body 205. The condyle portions 207 are therefore connected to each other by means of the connection portion 209. The fixing portion 203 is arranged here at a posterior end 210 of the connection portion 209. The fixing portion 203 is located mediolaterally between the two condyle portions 207. In the present case, the fixing portion 203 is located mediolaterally between the two receiving bores 201. One of the receiving bores 201 in each case can be present in the region of one of the condyle portions 207. Alternatively or in addition, the fixing portion 203 is arranged at a proximodistal distance from the two receiving bores. In the present case, the fixing portion 203 is arranged both mediolaterally between the two receiving bores 201 and also at a proximodistal distance from the two receiving bores 201. The fixing portion 203 forms an undercut 211 with respect to a posterior direction P. The undercut 110 of the fixing element 102 is therefore designed complementing the undercut 211 of the fixing portion 203.

In the present case, at least one of the receiving bores 201 is designed as a blind hole 212. In the present case, both of the receiving bores 201 are each formed as a blind hole 212. A bottom 213 of the blind hole 212 functions as a posterior stop for the positioning pins 101. Accordingly, the bottom 213, together with the first ends 107 of the positioning pins 101, establishes an anteroposterior position of the surgical instrument 1 relative to the trial implant 200.

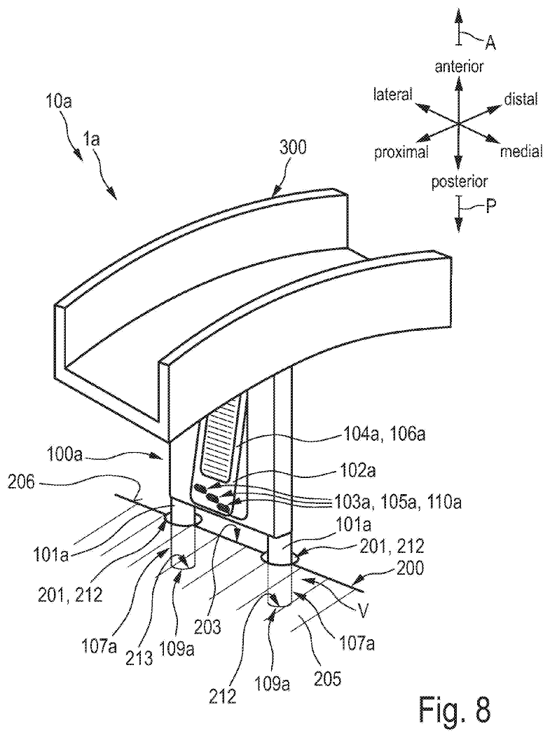

The further embodiment of the surgical instrument 1a according to FIG. 8 differs from the embodiment according to FIGS. 1 to 7 in the design of the fixing element 102a. The latter comprises a beam spring 104a instead of the torsion spring 104. The beam spring 104a has a tongue-shaped design. Moreover, according to FIG. 8, several, specifically three, form-fitting portions 103a are present. The form-fitting portions 103a are designed as snap-in hooks 105a. Each of the form-fitting portions 103a forms an undercut 110a with respect to the anterior direction A. By means of the form-fitting portions 103a, three latching positions of the surgical instrument 1a can be established in which the fastening device 100a can latch onto the trial implant 200 in the anteroposterior direction. Otherwise, as regards the embodiment according to FIG. 8, reference is made to the preceding description of the embodiment according to FIGS. 1 to 7; the corresponding reference signs in FIG. 8 are suffixed by the letter a.

As has already been discussed, the surgical instrument system 10, 10a comprises the surgical instrument 1, la and also the femoral trial implant 200. The surgical instrument 1, la is fastened releasably to the trial implant 200. The positioning pins 101, 101a and the receiving bores 201 are designed complementing each other. The positioning pins 101, 101a are plugged at least partially into the receiving bores 201 in the posterior direction P. The fixing element 102, 102a and the fixing portion 203 are tailored to each other to produce the form fit S. The surgical instrument 1 is held on the trial implant 200 by means of the form fit S. The form fit S holds the surgical instrument 1, la in the anterior direction A on the trial implant 200. Between the bottoms 213 of the blind holes 212 and the first ends 107 of the positioning pins 101, a form fit can be produced which holds the surgical instrument 1, la in the posterior direction P on the trial implant 200. Moreover, between the positioning pins 101 and the associated receiving bores 201, a form fit can be produced which holds the surgical instrument 100, 100a in the proximodistal direction and mediolateral direction on the trial implant 200.

The surgical instrument system 10, 10a comprises a plurality of trial implants 200. The trial implants 200 differ in terms of their size. The trial implants 200 of different sizes are adapted to femoral bones of different sizes. Depending on the anatomy of the treated patient, a suitable trial implant 200 can therefore be selected. In all of the trial implants 200, the two receiving bores 201 are arranged at a proximodistal distance 11 from a proximally outermost point DP of the respective trial implant 200. This point DP can be what is called a dwell point. In the present case, the distance 11 is related to the central longitudinal axes of the receiving bores 201. The proximodistal distance 11 is the same, i.e. identical, in all of the trial implants 200. Moreover, the receiving bores 201 of each of the trial implants 200 are arranged at a mediolateral distance 12 from one another. The mediolateral distance 12 is related for example to the central longitudinal axes of the receiving bores 201. The mediolateral distance 12 is the same, i.e. identical, in all of the trial implants 200. In other words, irrespective of their size, all of the trial implants 200 of the surgical instrument system 10, 10a have receiving bores 201 with a uniform proximodistal distance 11 and uniform mediolateral distance 12.

The surgical instrument system 10, 10a can moreover comprise a guide device 300. The guide device 300 can be connected releasably to the fastening device 100. A further fastening device can moreover be connected releasably to the guide device 300, on which further fastening device a tibial cutting block can be fastened for guiding a resection cut on the proximal tibia. By means of the guide device 300, the further fastening device can be aligned relative to the first fastening device 100, 100a. The releasable connection of the first fastening device 100, 100a to the guide device 300 can be realized by means of a plug connection 400.

The guide device 300 can have the shape of an arc of a circle, such that the fastening device 100, 100a is pivotably movable in a guided manner relative to the further fastening device. In this way, the inclination of the tibial cutting block in a guide plane can be adjusted. The guide plane has a sagittal orientation and thus has an anteroposterior and proximodistal extent. A pivot axis about which the two fastening devices are pivotably movable relative to each other by means of the guide device preferably intersects a mechanical tibial axis in the sagittal guide plane. Said inclination is also referred to as posterior or anterior slope. The adjustability of the inclination of the tibial cutting block (hereinafter slope for short) on the one hand permits adaptation to parameters that have been established before the operation. On the other hand, an actual flexion or extension position of the leg can be taken into account and compensated by the mobility of the guide device. It is thereby possible to ensure that an adjustment of the slope does not at the same time lead to an unwanted change of a proximodistal position of the tibial cutting block and thus of a so-called tibial cutting height.

1. A surgical instrument for use in a knee joint replacement operation, the surgical instrument comprising a fastening device configured for releasably fastening to a femoral trial implant mounted on a distal femur, the fastening device comprising two positioning pins which are arranged spaced apart mediolaterally from each other and which, for the purpose of positioning the fastening device, are designed to be able to be plugged in a posterior direction into two receiving bores, complementary to the positioning pins, of the trial implant, the fastening device further comprising at least one fixing element for fixing the fastening device in a form-fitting manner to the trial implant in an anterior direction.

2. The surgical instrument according to claim 1, wherein the fixing element comprises at least one snap-in hook.

3. The surgical instrument according to claim 1, wherein the fixing element is adjustably movable, relative to the positioning pins, between a fixing position and a release position.

4. The surgical instrument according to claim 3, wherein the fastening device comprises at least one spring element for generating a spring force which biases the fixing element toward the fixing position.

5. The surgical instrument according to claim 4, wherein the at least one spring element comprises a torsion or beam spring element.

6. The surgical instrument according to claim 1, wherein the fixing element has at least one form-fitting portion for producing a form fit between the fastening device and the trial implant.

7. The surgical instrument according to claim 6, wherein the positioning pins are each elongate in an anteroposterior direction between a first and a second end, wherein a first end portion present at the respective first end is receivable in the posterior direction in one of the receiving bores of the trial implant so as to form a plug connection.

8. The surgical instrument according to claim 7, wherein the at least one form-fitting portion creates an undercut with respect to the anterior direction.

9. The surgical instrument according to claim 8, wherein the first ends are exposed posteriorly and the undercut is exposed anteriorly, such that the trial implant can be positioned anteroposteriorly between the first ends and the undercut.

10. A femoral trial implant for use in a knee joint replacement operation, the femoral trial implant comprising:
a bone contact surface configured for contacting a distal femur;
two receiving bores each configured to receive a positioning pin of a surgical instrument; and
a fixing portion which is configured for form-fit connection to a fixing element of the surgical instrument.

11. The femoral trial implant according to claim 10, wherein the bone contact surface is arranged on a proximal rear face of a main body of the trial implant and lies opposite a distal front face of the main body, wherein the main body has two condyle portions which are spaced mediolaterally apart from each other and which each extend in an anteroposterior direction and transition at their anterior ends into a connection portion of the main body, wherein the fixing portion is arranged at a posterior end of the connection portion, mediolaterally between the two condyle portions.

12. The femoral trial implant according to claim 10, wherein the fixing portion is arranged mediolaterally between the two receiving bores and/or at a proximodistal distance from the two receiving bores.

13. The femoral trial implant according to claim 10, wherein the fixing portion creates an undercut with respect to a posterior direction.

14. The femoral trial implant according to claim 10, wherein at least one of the receiving bores is a blind hole.

15. A surgical instrument system for use in a knee joint replacement operation, the surgical instrument system comprising:
the surgical instrument according to claim 1; and
at least one femoral trial implant comprising:
a bone contact surface configured for contacting a distal femur;
two receiving bores each configured to receive one of the positioning pins of the surgical instrument; and
a fixing portion configured for form-fit connection to a fixing element of the surgical instrument,
wherein the surgical instrument is releasably fastened to the at least one femoral trial implant,
wherein the positioning pins and the receiving bores have complementary configurations,
wherein the positioning pins are at least partially plugged into the receiving bores along the posterior direction,
wherein the fixing element and the fixing portion are tailored to each other to produce a form fit, and
wherein the surgical instrument is fixed on the at least one femoral trial implant by the form fit.

16. The surgical instrument system according to claim 15, wherein:
the at least one femoral trial implant comprises a plurality of femoral trial implants of different sizes,
the receiving bores of each femoral trial implant are arranged at an identical proximodistal distance from a proximally outermost point of the femoral trial implant with respect to a central longitudinal axis of the femoral trial implant, and
the receiving bores of each femoral trial implant are arranged at an identical mediolateral distance from each other with respect to the central longitudinal axis.
